(19) United States
(12) Patent Application Publication
(10) Pub. No.: US 2006/0161238 A1
(43) Pub. Date: Jul. 20, 2006

(54) Thoracoscopic epicardial cardiac lead with guiding deployment applicator and method therefor

(76) Inventor: Jeffrey A. Hall, Hoover, AL (US)

Correspondence Address: Schwegman, Lundberg, Woessner & Kluth, P.A., P.O. Box 2938, Minneapolis, MN, 55402, US

(21) Appl. No.: 11/196,781

(22) Filed: Aug. 3, 2005

Related U.S. Application Data
(60) Provisional application No. 60/598,323, filed on Aug. 3, 2004.

(51) Int. Cl. A61N 1/05

(52) U.S. Cl. 607/129

(57) ABSTRACT

An epicardial lead assembly includes a thoracoscopic epicardial lead including an electrode, a guiding applicator, and, optionally, an introducer. The epicardial lead includes therapy delivering leads such as, but not limited to, pacing leads, and combination pacing-defibrillation leads.

### CROSS-REFERENCE TO RELATED APPLICATION(S)

This application claims the benefit of U.S. Provisional Application No. 60/598,323, filed on Aug. 3, 2004, under 35 U.S.C. § 119(e), which is hereby incorporated by reference.

### TECHNICAL FIELD

The present application relates generally to the field of medical leads. More particularly, it pertains to epicardial cardiac leads and a guiding deployment applicator.

### BACKGROUND

Leads implanted in or about the heart have been used to reverse certain life threatening arrhythmia, or to stimulate contraction of the heart. Electrical energy is applied to the heart via the leads to return the heart to normal rhythm. For example, one technique is to implant the electrode transvenously through the coronary sinus to reach a location below the left atrium, and then apply electrical energy. However, for some patients, a transvenous lead is not an option if, for example, the coronary sinus is inaccessible due to prior lead placement or anatomical anomalies. The coronary sinus ostium also can be difficult to locate. Furthermore, the transvenous lead is not an option for some patients with an artificial tricuspid valve. While it is possible to access the heart thoracoscopically, traditional thorascospic epicardial leads require multiple sites on the patient to be accessed by various instruments, for example, as shown in U.S. Pat. No. 5,871,532.

Accordingly, what is needed is a way in which to access the heart thoracoscopically at a single site.

### SUMMARY

An epicardial lead assembly includes a thoracoscopic epicardial lead including an electrode, a guiding applicator, and optionally an introducer. The epicardial lead includes therapy delivering leads such as, but not limited to, pacing leads, and combination pacing-defibrillation leads.

Several options for the lead assembly are as follows. For example, in one option, the lead includes deployable staple arms. In another option, the lead includes staples formed of absorbable material. In yet another option, the lead assembly includes an introducer with visualization features, and/or sealing features, allowing for a seal to be created during the implant process.

A method includes thoracoscopically accessing a least a portion of a heart at a thoracic entrance having a single access port, advancing an epicardial electrode through the single access port, attaching the epicardial electrode to tissue associated with the heart, and applying signals to the epicardial electrode.

Several options for the method are as follows. For example, in one option, the method includes forming a vacuum seal at the thoracic entrance. In another option, the method includes deploying staple arms from a position within the electrode to an extended position, for example, by rotating the staple arms out from the epicardial electrode.

The epicardial lead and methods, as described above and below, provide a minimally invasive manner in which to thoracoscopically place an epicardial pacing lead, for example, where the epicaridial lead is placed using a single point of entry into the patient. Furthermore, the left ventricular wall could be accessed via numerous approaches from the anterior, anterior-lateral or lateral thorax to subxyphoid. The addition of a deflectable guide within the pericardial space would separate the entrance point through the chest and pericardium from the final lead placement point which may be difficult to predict. This would also allow the surgeon to chose the optimum anatomical entrance point into the thorax and pericardium. Additionally, the embodiment involving pressure isolation might overcome the objection to external epicardial lead placement in the creation of a pneumothorax and dropping of a lung.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 is a block diagram illustrating an epicardial lead system constructed in accordance with at least one embodiment.

FIG. 2A is an end view illustrating a portion of an epicardial lead constructed in accordance with at least one embodiment.

FIG. 2B is an end view illustrating a portion of an epicardial lead constructed in accordance with at least one embodiment.

FIG. 2C is an end view illustrating a portion of an epicardial lead constructed in accordance with at least one embodiment.

FIG. 2D is an end view illustrating a portion of an epicardial lead constructed in accordance with at least one embodiment.

FIG. 3 is a side view illustrating a portion of an epicardial lead constructed in accordance with at least one embodiment.

FIG. 4 is a perspective view illustrating an assembly constructed in accordance with one embodiment.

FIG. 5 is a block diagram illustrating a portion of an epicardial lead constructed in accordance with one embodiment.

FIG. 6 is a side view illustrating a portion of an epicardial lead constructed in accordance with one embodiment.

FIG. 7 is a side perspective view of an applicator for an epicardial lead a portion of an epicardial lead constructed in accordance with one embodiment.

FIG. 8 is a block diagram illustrating a method in accordance with at least one embodiment.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

An epicardial lead assembly includes a thoracoscopic epicardial lead, a guiding applicator, and, optionally, an introducer. The epicardial lead includes therapy delivering leads such as, but not limited to, pacing leads, and combination pacing-defibrillation leads, etc, as will be further described below. The assembly allows for use of minimally invasive techniques to thoracoscopically place the lead with a single thoracoscopic port.

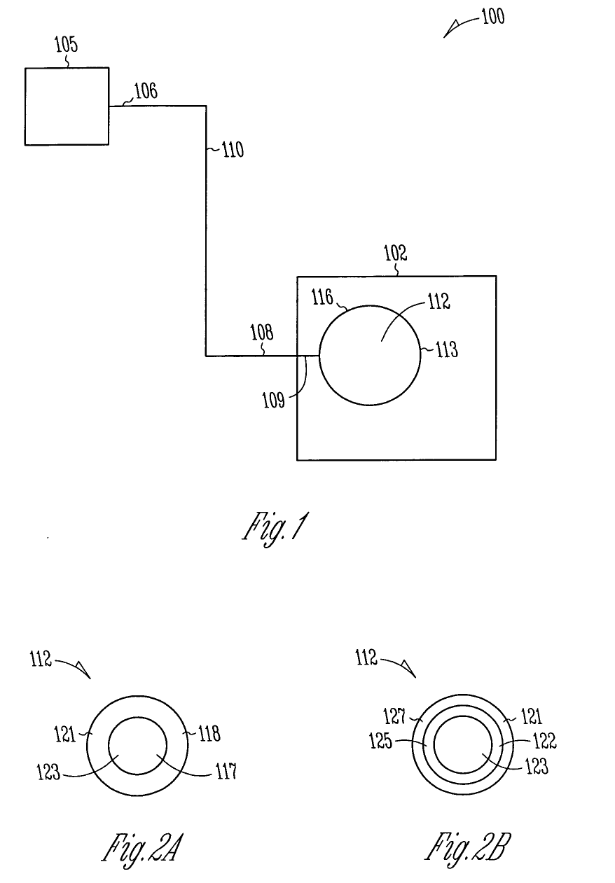

FIG. 1 illustrates one example of an epicardial lead assembly 100, shown in a block diagram form, where the epicardial lead assembly 100 delivers and/or receives electrical pulses or signals to stimulate, shock, and/or sense the heart 102. The epicardial lead assembly 100 includes an energy source, such as a pulse generator 105, and an epicardial lead 110. The pulse generator 105 includes a source of power as well as an electronic circuitry portion. The pulse generator 105, in one option, is a battery-powered device which generates a series of timed electrical discharges or pulses. The pulse generator 105 is generally implanted into a subcutaneous pocket made in the wall of the chest. Alternatively, the pulse generator 105 is placed in a subcutaneous pocket made in the abdomen, or in other locations. It should be noted that while the lead assembly 100 is illustrated for use with a heart, the lead assembly 100 is suitable for other forms of stimulation as well.

The epicardial lead 110 includes a lead body which extends from a proximal end 106, where it is coupled with the pulse generator 105. The epicardial lead 110 extends to a distal end 109, which is coupled with a portion of a heart 102, when implanted or otherwise coupled therewith, for example, epicardially, as further described below. Disposed along a portion of the lead body, for example, near the distal end 109 of the epicardial lead 110 includes at least one electrode assembly 116 which electrically couples the epicardial lead 110 with the heart 102. At least one electrical conductor 108 is disposed within the epicardial lead 110 and extends, in one option, from the proximal end 106 to the distal end 109 of the epicardial lead 110. The at least one electrical conductor 108 electrically couples the electrode assembly 116 with the pulse generator 105. The electrical conductors carry electrical current to and from the heart 102, and carry pulses between the pulse generator 105 and the electrode assembly 116.

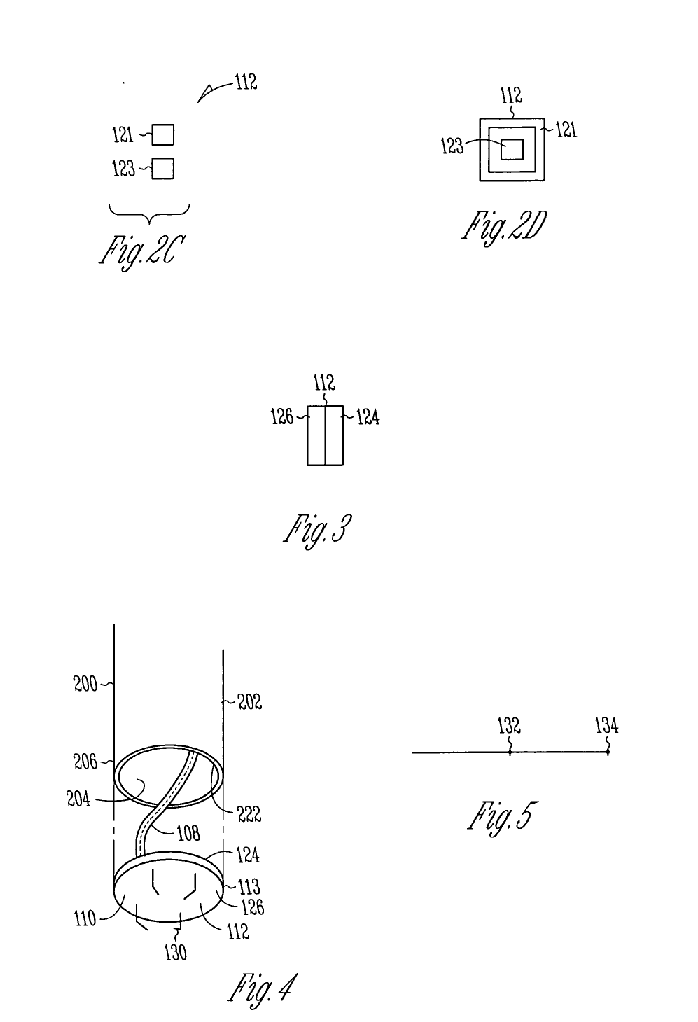

The electrode assembly 116 of the epicardial lead 110 includes an electrode disc 112, where the disc 112, in one option, includes a first electrode 121 and a second electrode 123 (FIGS. 2A-2D). The electrode disc 112, in one option, is a circular disc defined in part by an outer perimeter 113, however, the disc 112 is not limited to a circular shape. The electrode disc 112 assists in providing the electrical connection between the conductor 108 and the heart 102, for example, by epicardial placement of the electrode disc 112.

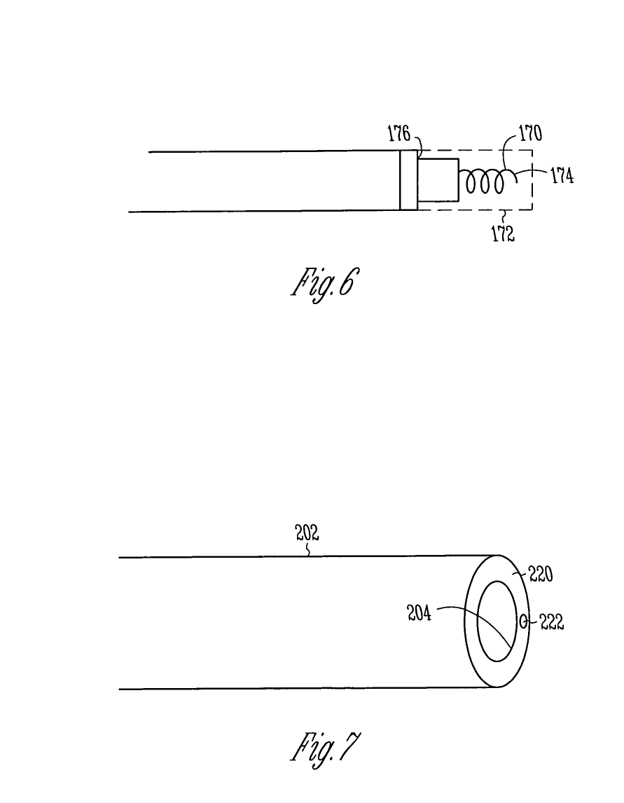

Referring to FIGS. 2A-2D, and 3, the variations for the electrode disc 112 are shown in greater detail. The electrode disc, in one option, includes one or more concentric electrode rings. For example, in one option, the epicardial lead is bipolar with a cathode center electrode 117, as shown in FIG. 2A, that is at least partially surrounded by a circumferential anode 118. In another option, the circumferential anode completely surrounds the perimeter of the cathode center electrode 117.

In yet another option, there are two or more electrodes 121, 123 that are spaced apart, such that there is a space 122 therebetween, for example, as illustrated in FIG. 2B. In one option, a first electrode 121 is spaced apart from a second electrode 123. In yet another option, multiple rings or partial rings allow for multipolar configurations. In another option, a non-reactive biomaterial collar 125 is surrounded by an attachment area 127, including, for example, but not limited to, staple arms, as discussed further below. In one option, the collar 125 includes a steroid material, dexamethasone as an acetate or phosphate or both, or any combination of these, thereby minimizing threshold rise and exit block.

The relative size and shape of the above-discussed electrodes would, in one option, be optimized for pacing and sensing characteristics along with minimizing the occurrence of exit block through stimulation of fibrous scar tissue around the electrode. For example, the electrodes and the backing or support material (discussed below) would have an optimum shape and surface to minimize risk of motion, movement, friction on epicardial vessels, orientation change (flipping with the pericardium). For example, the electrodes 121, 123 could be formed into concentric shapes, such as that shown in FIG. 2A, 2B, or 2D. Other shapes and/or configurations are possible, for example, as shown in FIG. 2C. The electrodes 121, 123 have, in one option, substantially equal surface area. In another option, the electrodes 121, 123 do not have substantially the same surface area.

In another option, as illustrated in FIG. 3, the electrode disc 112 includes an electrode surface 126, and at least one electrode backing material 124. The electrode backing material 124, in one option, is associated with a medicament. For example, in one option, the backing material 124 is impregnated with a cortiocosteroid or combination of corticosteriods such as, but not limited to, dexamethasone sodium phosphate or dexamethasone acetate. The backing material 124 is formed of a biocompatible material, for example, a Dacron® mesh, silicone, or PTFE. In another option, the electrode backing material 124 is made of electrically insulative material.

In yet another option, the electrode surface 126 includes a metallic ring, or a partial metallic ring. Alternatively, or in addition to the rings, the electrode surface 126 is formed of materials having increased surface area including, but not limited to, sputtered metallic material, sintered woven platinum, or other metallic mesh material.

The assembly 100 (FIG. 1) includes fixation features. The fixation features method may or may not be incorporated into the electrode area. For example, one electrode may be a fixation staple or similar fixation material, as discussed below. In another option, as illustrated in FIG. 5, there is fixation with the electrode 134, and a second electrode 132 and fixation point to provide strain relief.

The electrode disc 112 (FIGS. 2A-2D) includes one or more fixation features. For example, the fixation features include the electrode surface 126 having increased surface area, as discussed above. The fixation features are, optionally, disposed a distance from the electrodes to minimize the occurrence of exit block from tissue reaction and fibrous tissue proliferation. In one example of a fixation feature, the electrode disc 112 includes a porous structure, such as mesh. The fixation features, in one option, include staples, sutures, adhesives, or barbs.

In another option, the fixation features include curved barbs so that rotation of the disc sets the fixation. The barbs would engage the epicardium and fix on the tissue with rotation of the disc. The barbs optionally have a curved and downward orientation clockwise, for example, to engage and enter the myocardium when rotated clockwise. In another option, the electrode disc 112 includes one or more staple arms 130, as illustrated in FIG. 4.

The one or more staple arms 130 are mechanically coupled with the electrode disc 112, for example, 2-6 staple arms are coupled with the disc 112, optionally, around the circumference 113. The staple arms 130, in one option, are formed of a metal or otherwise electrically conductive material. For example, the staple arms 130 serve as an electrode to the assembly 100 (FIG. 1), and/or an extension of the electrodes. In another embodiment, the staple arms 130 are formed of an absorbable material such as, but not limited to, polygluconate acetate (PGA absorbable material through hydrolysis). In yet another option, a biomaterial collar is disposed between the electrode and the staple arms 130. The separation by the collar minimizes the occurrence of exit block by separating the electrode tissue interface from the area of tissue fixation. The staple arms 130, in one option, are electrically inactive.

The staple arms 130 are, in one option, mechanically coupled with the disc, and, in another option, are deployable from a first retracted position to a second extended position. For example, in one option, the staple arms 130 rotate out from the surface 126 of the electrode disc 112. For example, two concentric tubes are counter rotated to deploy the arms 130 to an extended position. In yet another option, the fixation feature is deployable remotely by the applicator outside of the thoracic cavity. For example, rotation of one of the tubes extends the arms into the myocardium, for example, the exterior tube. The interior tube operates as a fulcrum or rotation point.

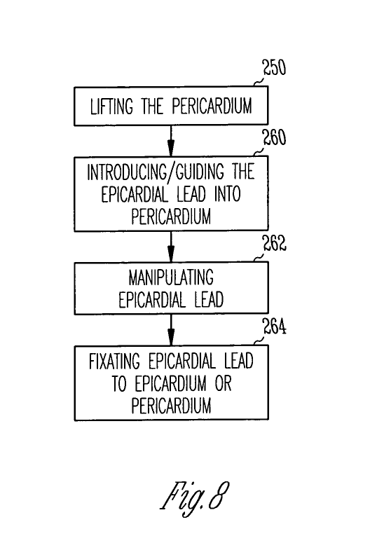

FIG. 6 illustrates another example of fixation features and method of application, where multiple tubes such as concentric tubes can be used to fixate the epicardial electrode disc. For example, an exposed helix 170 would be temporarily covered by a tube 172, that is retractable to expose the helix 170. The helix 170, in one option, is relatively short and thick compared to conventional endocardial leads. The helix 170, in one option, forms the cathode 174 and is buried in the myocardium after fixation. The anode 176 would be positioned substantially on the surface of the myocardium after implantation.

In yet another option, the staple arms 130 (see FIG. 4) are coupled with the disc 112 and are disposed in a curved shape. The curved shape of the staple arms 130 allow for rotating the disc and engaging the epicardium to fix the tissue. The curved shape of the arms 130 and the method of attaching the disc 112 would be helpful, for example, for patients who have undergone previous CABG and have portions of a pericardium that are missing and/or scarred. A further option, an additional fixation feature is disposed between an edge of the pericardial incision and the pacing lead, allowing strain relief for the lead, for example, as illustrated in FIG. 5.

The lead assembly 100 optionally includes the applicator 200, as illustrated in FIGS. 4 and 7. The applicator 200 is defined in part by an outer wall 202 and a central lumen 204 which each extend to the applicator distal end 206. The central lumen 204 is sized to receive, guide, and pass therethrough the electrode disc 112, including the various embodiments of disc 112, discussed above. In one option, a small spring coil or material shape is used to deploy the electrode disc 112 from an applicator 200 after introduction into the pericardium.

In one option, the applicator 200 includes visualization equipment, such as a light source 220, or a fiber optic light source. In another option, the applicator 200 further includes a viewing channel 222. The light source and/or the viewing channel are, in one option, formed in the outer wall 202 of the applicator 200, for example, by overmolding or co-extrusion. The light source 220, in an option, forms a ring within the wall 202. Alternatively, the light source 220 and the viewing channel 222 are segmented and placed around the circumference of the wall. In another option, the visualization features are disposed between two concentric tubes, that are, optionally, used to deploy fixation features. The visualization equipment assists in placement of the epicardial lead around vascular structures. In yet another embodiment, the light source 220 and the viewing channel 222 retract back over the applicator and/or through the lumen 204.

For example, as illustrated in FIG. 7, the viewing channel 222, such as the fiber optic channel is disposed about the circumference of the tubing structure shown. The viewing channel 222 is incorporated into the wall of the applicator 200. In one option, the wall of the applicator 200 is formed into a suitable thickness to incorporate the viewing channel 222 therein. The viewing channel 222 is, optionally, movable relative to the application 200, allowing for the retraction of the channel 222 and/or light source of the applicator 200. In yet another option, the viewing channel 222 is disposed within the lumen 204 of the applicator 200. Further options for the applicator allow for the applicator 200 to be steerable or flexible. For example, the applicator 200 includes one or more flexing wires, allowing for the applicator to be manipulated into position within the epicardium.

The applicator 200 further, optionally, includes an introducer. The introducer facilitates entrance into the pericardial cavity and allows for manipulation and deployment of the lead into the desired position on the epicardium. The introducer extends to a distal end and includes an introducer lumen therein. The introducer lumen is sized and configured to receive the applicator therein. The applicator is slidably received within the introducer. In one option, the introducer includes a conformal soft tip and suction.

Through use of the introducer, the pericardium is lifted, for example, with applied suction and pulled inside the introducer. The pericardium is, in one option, incised or punctured and dilated within the introducer. For example, a vacuum is applied to the introducer forming a seal with the tissue to lift the tissue. The introducer can further, optionally, be provided with structure such as teeth, similar to forceps, to cut or pierce the tissue as the tissue is pulled within the introducer.

In another option, edges of the introducer mechanically grasp one or more pericardial edges, allowing access to the pericardial space and the epicardium. The deflectable pacing lead guide is inserted, and allows movement of the pacing lead and electrodes within the pericardium with or without visualization via fiber options to define an optimum pacing location for the left ventricular pacing lead.

With use of the assembly 100, the physician can access the myocardium using a single port, instead of using multiple ports. FIG. 8 illustrates a block diagram of a method of using the assembly, described above.

The method includes lifting the pericardium, at 250. The pericardial entrance site is visualized, for example, with the integrated visualization features, as discussed above. The pericardium tissue lifted is, optionally, held by the introducer, and is further, optionally, opened, for example, incised or punctured, and dilated within the protection of the introducer tube.

The introducer lifts the tissue, for example, by providing a vacuum to the tissue, and puncture the tissue with structure incorporated into, onto, or within the introducer. Once the suction is applied, the pericardium is pulled inside the introducer, the pericardium is lifted off of the heart, and the pericardium is incised or punctured. The pericardium is dilated within the protection of the introducer to allow insertion therein of the applicator, and further for the placement of the epicardial lead therein. In another option, an external device surrounds the insertion device, where the external device incorporates conformal pressure seals on the skin to isolate the puncture, and reduce the need to drop the lung, such as the left lung. An external vacuum seal would surround the thoracic entrance point to eliminate pneumothorax by maintaining vacuum during entrance, manipulation and closure. With a small flexible system, the lead could be placed without dropping a lung lobe and closure would be simplified.

In another option, the pericardium could be lifted with grasping arms of the introducer and/or guide, for example, that rotate outward from a tip of the introducer. The arms grasp the pericardium, and the introducer is retracted to lift the pericardium. The pericardium can be opened using, for example, a device such as pinpoint electrocautery.

The applicator is inserted through the introducer, and the epicardial lead is advanced, for example, through the applicator at 260. The applicator is used to guide the epicardial lead within the pericardial space, 262, with or without visualization to define an optimum pacing location for the left ventricular pacing lead. The pacing lead guide, optionally, includes a rail therealong to further guide the lead. The guide is, optionally, further manipulated within the pericardium to allow for a large range of lead placement, for example, through use of a steering wire. In another option, the guide is telescoped into place within the pericardial space.

The epicardial lead is attached to tissue, for example, to the epicardium, at 264. For example, the epicardial lead is attached using one or more of the fixation features, discussed above. Examples of fixation include deploying the staple arms or rotating the lead to attach fixation features, such as curved arms, to attach the lead to the tissue. Other suitable attachment fixation methods include, but are not limited to, staples, sutures, or adhesives, where the attachment can be automatic, or non-automatic. Fixation can occur at a distance from the electrodes to minimize the occurrence of exit block from tissue reaction and fibrous tissue proliferation. An additional fixation method could occur between the edge of the pericardial incision and the pacing lead, giving an additional fixation point, allowing strain relief for the pacing lead. After the fixation occurs, electronic signals are sent via the lead to the tissue to deliver therapy and/or to monitor the heart. In another option, the lead is pericardially attached, instead of epicardially, which may lower damage to vessels, and may allow for a faster procedure. The location of the pericardial entrance point may set the relative location of the lead. As the distance from the pericardial entrance point to the electrode increases, the need for epicardial fixation increases as the arc of electrode movement within the pericardium proportionally increases.

The epicardial lead and methods as described above provide a minimally invasive manner in which to thoracoscopically place an epicardial pacing lead. This further assists in left ventricular lead placement in patients with inaccessible coronary sinuses due to, for example, prior lead placement or anatomical anomalies. Furthermore, epicardial approaches may be necessary in patients with high LV thresholds by coronary sinus access, and in patients with artificial tricuspid valves, precluding them from a lead passing through the valve. Having a single thoracoscopic port allows for additional access locations, and provides fast and simple placement for the epicardial lead. For example, the left ventricular wall could be accessed via numerous approaches from the anterior, anterior-lateral or lateral thorax to subxyphoid.

The addition of a deflectable guide within the pericardial space would separate the entrance point through the chest and pericardium from the final lead placement point which may be difficult to predict. This would also allow the surgeon to chose the optimum anatomical entrance point into the thorax and pericardium. Additionally, the embodiment involving pressure isolation might overcome the objection to external epicardial lead placement in the creation of a pneumothorax and dropping of a lung.

It is to be understood that the above description is intended to be illustrative, and not restrictive. Many other embodiments will be apparent to those of skill in the art upon reading and understanding the above description. It should be noted that embodiments discussed in different portions of the description or referred to in different drawings can be combined to form additional embodiments of the present application. The scope of the invention should, therefore, be determined with reference to the appended claims, along with the full scope of equivalents to which such claims are entitled.

What is claimed is:

1. An epicardial lead assembly comprising:
at least one epicardial electrode disc having one or more electrodes associated therewith;
the at least one electrode disc coupled to an energy source;
at least one applicator extending from a proximal end to a distal end;
the at least one disc having a first position within the distal end, and a second position extended from the distal end of the applicator; and
wherein the epicardial electrode disc includes one or more electrodes formed thereon.

2. The epicardial lead assembly as recited in claim 1, further comprising one or more staples mechanically coupled with the electrode disc.

3. The epicardial lead assembly as recited in claim 2, wherein the one or more electrode rings are spaced away from the one or more staples.

4. The epicardial lead assembly as recited in claim 2, wherein the one or more staples are deployable from a first retracted position to a second extended position.

5. The epicardial lead assembly as recited in claim 2, wherein the one or more staples are electrically inactive.

6. The epicardial lead assembly as recited in claim 2, wherein the one or more staples are formed of absorbable material.

7. The epicardial lead assembly as recited in claim 1, further comprising backing material impregnated with at least one cortiocosteroid.

8. The epicardial lead assembly as recited in claim 1, further comprising a visualization introducer.

9. An epicardial lead assembly comprising:
at least one electrode disc having one or more electrodes associated therewith;
the at least one electrode disc electrically coupled to an energy source;
wherein the electrode disc includes one or more electrode rings formed thereon; and
means for accessing an epicardial portion of a heart from a single thoracoscopic entrance.

10. The epicardial lead assembly as recited in claim 9, wherein the means for accessing an epicardial portion of a heart from a single thoracoscopic entrance includes means for attaching the electrode disc at a single site of tissue.

11. The epicardial lead assembly as recited in claim 9, further comprising one or more staple arms coupled with the electrode disc.

12. The epicardial lead assembly as recited in claim 11, wherein the staple arms have a curved shape.

13. The epicardial lead assembly as recited in claim 11, wherein the staple arms are bioabsorbable.

14. A method comprising:
thoracoscopically accessing a least a portion of a heart at a thoracic entrance having a single access port;
advancing an epicardial electrode through the single access port with an applicator, the epicardial electrode including a porous structure;
attaching the epicardial electrode to tissue associated with the heart; and
applying signals to the epicardial electrode.

15. The method as recited in claim 14, wherein attaching the epicardial electrode includes attaching the epicardial electrode to at least a portion of the pericardium.

16. The method as recited in claim 14, wherein attaching the epicardial electrode includes attaching the epicardial electrode to at least a portion of the epicardium.

17. The method as recited in claim 14, further including forming an external vacuum seal surrounding the thoracic entrance prior to attaching the epicardial electrode.

18. The method as recited in claim 14, further comprising deploying the epicardial electrode with a spring mechanism.

19. The method as recited in claim 14, further comprising deploying staple arms out of the epicardial electrode, and attaching the electrode to the tissue includes engaging the staple arms with the tissue.

20. The method as recited in claim 19, wherein deploying the staple arms includes rotating the staple arms out from the epicardial electrode.

21. A method comprising:
thoracoscopically accessing a least a portion of a heart at a thoracic entrance having a single access port;
advancing a disc-shaped epicardial electrode through the single access port with an applicator;
attaching the disc-shaped epicardial electrode to tissue associated with the heart; and
applying signals to the epicardial electrode.

22. The method as recited in claim 21, further comprising visualizing a portion of the heart with a visualization assembly coupled with the applicator.

23. The method as recited in claim 22, further comprising retracting the visualization assembly away from an end portion of the applicator.

24. The method as recited in claim 21, further comprising applying suction with an introducer, and lifting a portion of a pericardium.
